(19) United States
(12) Patent Application Publication
(10) Pub. No.: US 2007/0020627 A1
(43) Pub. Date: Jan. 25, 2007

(54) Artificial transcription factors

(75) Inventor: Carlos F Barbas III, Solana Beach, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/514,763

(22) PCT Filed: Jun. 6, 2003
(86) PCT No.: PCT/US03/17946

§ 371 Date: Jul. 27, 2006

Related U.S. Application Data
(60) Provisional application No. 60/388,055, filed on Jun. 11, 2002.

(51) Int. Cl. C12Q 1/68; C07H 21/04; C12P 21/06; C12P 19/34; C12N 9/22

(52) U.S. Cl. 435/6; 435/69.1; 435/320.1; 435/325; 435/199; 536/23.2; 435/91.2

(57) ABSTRACT

Transcription regulating polypeptides that contain a plurality of DNA binding domains are provided. The polypeptides optionally contain one or more transcription regulating domains. Polynucleotides that encode the polypeptides and use of the polypeptides and polynucleotides are also provided.

### CROSS-REFERENCE TO RELATED APPLICATIONS

This patent is a continuation-in-part of U.S. Provisional Patent Application Ser. No. 60/388,055, filed Jun. 11, 2002, the disclosure of which is incorporated herein by reference.

### TECHNICAL FIELD OF THE INVENTION

The field of this invention is gene transcription. More particularly, this invention provides a gene transcription regulating polypeptide that contains a plurality of DNA binding domains directed to different target nucleotide sequences within one or more genes.

### BACKGROUND OF THE INVENTION

The construction of artificial transcription factors has been of great interest in the past years. Gene expression can be specifically regulated by polydactyl zinc finger proteins fused to regulatory domains (See, e.g., U.S. Pat. Nos. 6,242,568; 6,140,466; and 6,140,081, the disclosures of which are incorporated herein by reference).

Zinc finger domains of the Cys₂-His₂ family have been most promising for the construction of artificial transcription factors due to their modular structure. Each domain consists of approximately 30 amino acids and folds into a ββα structure stabilized by hydrophobic interactions and chelation of a zinc ion by the conserved Cys₂-His₂ residues. To date, the best characterized protein of this family of zinc finger proteins is the mouse transcription factor Zif 268 [Pavletich et al., (1991) Science 252(5007), 809-817; Elrod-Erickson et al., (1996) Structure 4(10), 1171-1180]. The analysis of the Zif268/DNA complex suggested that DNA binding is predominantly achieved by the interaction of amino acid residues of the α-helix in position −1, 3, and 6 with the 3′, middle, and 5′ nucleotide of a 3 bp DNA subsite, respectively. Positions 1, 2 and 5 have been shown to make direct or water-mediated contacts with the phosphate backbone of the DNA. Leucine is usually found in position 4 and packs into the hydrophobic core of the domain. Position 2 of the α-helix has been shown to interact with other helix residues and, in addition, can make contact to a nucleotide outside the 3 bp subsite [Pavletich et al., (1991) Science 252(5007), 809-817; Elrod-Erickson et al., (1996) Structure 4(10), 1171-1180; Isalan, M. et al., (1997) Proc Natl Acad Sci U S A 94(11), 5617-5621].

Zinc finger DNA binding domains can be assembled into zinc finger proteins recognizing extended 18 bp DNA sequences which are unique within the human or any other genome. In addition, these proteins function as transcription factors and are capable of altering gene expression when fused to regulatory domains and can even be made hormone-dependent by fusion to ligand-binding domains of nuclear hormone receptors. To date, however, polypeptides containing one or more zinc finger binding domains target a single gene or contain a single transcription regulating domain. There is a need in the art, therefore, for transcription regulating polypeptides that can be used to target more than one gene or contain more than one transcription regulating domain.

The present disclosure provides polypeptides that contain a plurality of DNA binding domains and one or more transcription regulating domains. Such polypeptides can be used to regulate transcription of more than one target gene or to enhance the activation or repression of single genes.

### BRIEF SUMMARY OF THE INVENTION

The present invention provides a non-naturally occurring artificial transcription factor polypeptide comprising a plurality of DNA binding domains (DNB) operatively linked to each other. The DNA binding domains each bind independently to a same or different nucleotide sequence. The polypeptide can further contain one or more transcription regulating domains, each of which is operatively linked to one of the DNA binding domains.

The different nucleotide sequences are located in a transcriptional control region of the same gene or different genes. Where the nucleotide sequences are located in transcriptional control regions of a single gene, such nucleotide sequences are separated from each other by at least 10 base pairs. The polypeptide contains two or more DNBs. In one embodiment, the polypeptide contains two or three DNA binding domains. Each DNA binding domain preferably contains from 3 to 6 zinc finger peptides and, more preferably 6 zinc finger peptides.

The DNA binding domains are preferably operatively linked to each other with an amino acid residue sequence of from 5 to 50 amino acid residues, preferably from 5 to 40 amino acid residues, more preferably from 5 to 30 amino acid residues and, even more preferably from 5 to 15 amino acid residues.

In another aspect, the present invention provides a polynucleotide that encodes a polypeptide of this invention, an expression vector that contains such a polynucleotide and a cell transformed with such a polynucleotide or expression vector.

In yet another aspect, the present invention provides processes for regulating gene transcription. In one embodiment, a present method is directed to simultaneously regulating transcription of a plurality of DNA target genes in a cell. Such a method comprises the steps of transforming the cell with a polynucleotide that encodes a polypeptide having a plurality of operatively linked DNA binding domains, each of which DNA binding domains specifically binds to a nucleotide sequence in a transcriptional control region of different DNA target genes and maintaining the cell under conditions and for a period of time sufficient for expression of the polypeptide. In a second embodiment, a method is directed to regulating transcription of a single gene. Such a method comprises the steps of transforming the cell with a polynucleotide that encodes a polypeptide having a plurality of operatively linked DNA binding domains, each of which DNA binding domains specifically binds to a different nucleotide sequence in a transcriptional control region of the DNA target gene and maintaining the cell under conditions and for a period of time sufficient for expression of the polypeptide. Preferably, a method of this invention uses a polypeptide that also contains one or more transcription regulating domains.

### BRIEF DESCRIPTION OF THE DRAWINGS

In the drawings that form a portion of the specification,

FIG. 1 shows a schematic representation of a transcription factor polypeptide of this invention. DNB represents a DNA binding domain. N is from 1 to 10. ˜Represents an amino acid residue linker.

FIG. 2 shows exemplary arrangements of DNBs and repressor (SKD) or activation (VP64) transcription regulating domains assembled out of two DNBs connected by a flexible linker.

### DETAILED DESCRIPTION OF THE INVENTION

I. The Invention

The present invention provides non-naturally occurring transcription factor polypeptides useful for regulating gene transcription, polynucleotides that encode such polypeptides and the use of such polypeptides and polynucleotides in regulating gene transcription.

II. Polypeptides

The present invention provides non-naturally occurring polypeptides that contain a plurality of DNA binding domains (DNB), which binding domains are derived from zinc finger DNA binding peptides (See FIG. 1). A polypeptide of this invention is non-naturally occurring. As used herein, the term “non-naturally occurring” means, for example, one or more of the following: (a) a polypeptide comprised of a non-naturally occurring amino acid sequence; (b) a polypeptide having a non-naturally occurring secondary structure not associated with the polypeptide as it occurs in nature; (c) a polypeptide that includes one or more amino acids not normally associated with the species of organism in which that polypeptide occurs in nature; (d) a polypeptide that includes a stereoisomer of one or more of the amino acids comprising the polypeptide, which stereoisomer is not associated with the polypeptide as it occurs in nature; (e) a polypeptide that includes one or more chemical moieties other than one of the natural amino acids; or (f) an isolated portion of a naturally occurring amino acid sequence (e.g., a truncated sequence). A polypeptide of this invention exists in an isolated form and purified to be substantially free of contaminating substances. A polypeptide can be synthetic in nature. That is, the polypeptide is isolated and purified from natural sources or made de novo using techniques well known in the art. A polypeptide of this invention can be made using a variety of standard techniques well known in the art.

Amino acid residues of polypeptides are expressed herein using the standard 1 or 3-letter codes (See Table 1, below).

TABLE 1

| | 3 Letter | 1 Letter |
|---|---|---|
| Amino Acid | Code | Code |
| Alanine | Ala | A |
| Cysteine | Cys | C |
| Aspartic Acid | Asp | D |
| Glutamic Acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |
| Stop Codons | | Z |

In certain embodiments, a polypeptide variant comprises a conservatively substituted amino acid residue. It is preferred that each amino acid substitution is made by substituting the amino acid of interest with an amino acid from a group of similar amino acid(s) as listed in the Table 2, below. (See Biochemistry, 3rd Edition, Stryer, Freeman Publisher (1988) pages 16-40, incorporated herein by reference).

Referring to the Table 2, for example, in certain embodiments, a G amino acid residue in a desired polypeptide is substituted with an A, V, L, or I. In another example, an N residue in a desired polypeptide is substituted with a D, E, or Q. It is generally preferred that the first amino acid (or codon in the underlying polynucleotide) of an open reading frame is methionine.

TABLE 2
Preferred Amino Acid Grouping for Conservative Substitution

| | Conservative |
|---|---|
| Side Chain Characteristic | Amino Acid Groups |
| Aliphatic | G, A, V, L, I |
| Aliphatic with secondary amino group | P |
| Aromatic | F, Y, W |
| Sulfur containing | C, M |
| Aliphatic hydroxyl | S, T |
| Basic | K, R, H |
| Acidic | D, E, N, Q |

A DNA binding domain of an instant polypeptide is derived or isolated from zinc finger DNA binding peptides, which peptides are well known in the art. Preferably, the zinc finger DNA binding peptide is derived from a Cys₂-His₂ type zinc finger. A zinc finger DNA binding peptide derivative can be derived or produced from a wild type zinc finger protein by truncation or expansion, or as a variant of a wild type-derived peptide by a process of site directed mutagenesis, or by a combination of the procedures (See e.g., U.S. Pat. Nos. 6,242,568; 6,140,466; and 6,140,081, the disclosures of which are incorporated herein by reference). The term “truncated” refers to a zinc finger-nucleotide binding polypeptide that contains less that the full number of zinc fingers found in the native zinc finger binding protein or that has been deleted of non-desired sequences. For example, truncation of the zinc finger-nucleotide binding protein TFIIIA, which naturally contains nine zinc fingers, might be a polypeptide with only zinc fingers one through three. Expansion refers to a zinc finger polypeptide to which additional zinc finger modules have been added. For example, TFIIIA may be extended to 12 fingers by adding 3 zinc finger domains.

In addition, a truncated zinc finger-nucleotide binding polypeptide may include zinc finger modules from more than one wild type polypeptide, thus resulting in a “hybrid” zinc finger-nucleotide binding polypeptide. The term “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized. Examples of known zinc finger-nucleotide binding polypeptides that can be truncated, expanded, and/or mutagenized according to the present invention in order to inhibit the function of a nucleotide sequence containing a zinc finger-nucleotide binding motif includes TFIIIA and zif268. Other zinc finger-nucleotide binding proteins will be known to those of skill in the art.

A polypeptide of this invention comprises a plurality of DNA binding domains. Preferably, the polypeptide contains from 2 to 10 such domains, more preferably from 2 to 5 such domains and, most preferably, 2 or 3 such domains. The DNA binding domains are operatively linked to each other. By “operatively linked” is meant that the structure and function of each DNA binding domain is unaffected by the linking of any other such domain. In one embodiment, the DNA binding domains are directly linked or bonded together via well known peptide linkages. In another embodiment, the DNA binding domains are operatively linked using a peptide linker containing from 5 to 50 amino acid residues. Preferably, the linker contains from 5 to 40 amino acid residues, more preferably from 5 to 30 amino acid residues and, even more preferably from 5 to 15 amino acid residues. The linkers are preferably flexible. Exemplary such linkers are set forth below.

(SEQ ID NO:1)

Linker 1: TGEKP

(SEQ ID NO:2)

Linker 2: PGGGGSGGGGTGSSRSSSTGEKP

(SEQ ID NO:3)

Linker 3: PGSSGGGGSGGGGGGSTGGGSGGGGTGSSRSSSTGEKP

(SEQ ID NO:4)

Linker 4: TGGGGSGGGGTGEKP

Where a transcription factor polypeptide of this invention contains 2 DNA binding domains, a single linker operatively links those domains. Where more than two DNA binding domains are present, a linker is used to operatively link each binding domain. In such an embodiment, the same or different linker can be employed at each linking location.

DNA binding domains used in the present transcription factors can be naturally-occurring or non-naturally occurring. Naturally-occurring zinc finger DNA binding domains are well known in the art. In a preferred embodiment, at least one DNA binding domain of a present transcription factor is non-naturally occurring. Each of the DNA binding domains is preferably designed and made to specifically bind nucleotide target sequences corresponding to the formula 5′-NNN-3′, where N is any nucleotide (i.e., A, C, G or T). Such DNA binding domains are well known in the art (See. e.g. U.S. Pat. Nos. 6,242,568, 6,140,466 and 6,140,081, the disclosures of which are incorporated herein by reference). A zinc finger DNA binding peptide of this invention comprises a unique heptamer (contiguous sequence of 7 amino acid residues) within the α-helical domain of the peptide, which heptameric sequence determines binding specificity to a target nucleotide. That heptameric sequence can be located anywhere within the a-helical domain but it is preferred that the heptamer extend from position −1 to position 6 as the residues are conventionally numbered in the art. A peptide can include any β-sheet and framework sequences known in the art to function as part of a zinc finger peptide.

Previously we reported the characterization of 16 zinc finger domains specifically recognizing each of the 5′-GNN-3′ type of DNA sequences, that were isolated by phage display selections based on C7, a variant of the mouse transcription factor Zif268 and refined by site-directed mutagenesis [U.S. Pat. No. 6,140,081, the disclosure of which is incorporated herein by reference]. Briefly, phage display libraries of zinc finger proteins were created and selected under conditions that favored enrichment of sequence specific proteins. Zinc finger domains recognizing a number of sequences required refinement by site-directed mutagenesis that was guided by both phage selection data and structural information. A similar system has been employed to identify domains that recognize the 5′-TNN-3′ type of DNA sequences.

To extend the availability of zinc finger domains for the construction of artificial transcription factors, domains specifically recognizing the 5′-ANN-3′ and 5′-CNN-3′ types of DNA sequences were selected. Briefly, the helix TSG-N-LVR (SEQ ID NO: 5), previously characterized in finger 2 position to bind with high specificity to the triplet 5′-GAT-3′, containing finger 1 and 2 of C7 and the 5′-GAT-3′-recognition helix in finger-3 position, was analyzed for DNA-binding specificity on targets with different finger-2 subsites by multi-target ELISA in comparison with the original C7 protein [See, e.g., Dreier, B. et al.: J Biol Chem Aug. 3, 2001; 276(31):29466-78].

A polypeptide of this invention can further comprise one or more transcription regulating domains. A transcription regulating domain can be an activation domain or a repression domain, as is well known in the art. An exemplary repression domain peptide is the ERF repressor domain (ERD), (Sgouras, D. N., Athanasiou, M. A., Beal, G. J., Jr., Fisher, R. J., Blair, D. G. & Mavrothalassitis, G. J. (1995) EMBO J. 14, 4781-4793), defined by amino acids 473 to 530 of the ets2 repressor factor (ERF). This domain mediates the antagonistic effect of ERF on the activity of transcription factors of the ets family. A second repressor protein is prepared using the Krüppel-associated box (KRAB) domain (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). This repressor domain is commonly found at the N-terminus of zinc finger proteins and presumably exerts its repressive activity on TATA-dependent transcription in a distance- and orientation-independent manner (Pengue, G. & Lania, L. (1996) Proc. Natl. Acad. Sci. USA 93, 1015-1020), by interacting with the RING finger protein KAP-1 (Friedman, J. R., Fredericks, W. J., Jensen, D. E., Speicher, D. W., Huang, X.-P., Neilson, E. G. & Rauscher III, F. J. (1996) Genes & Dev. 10, 2067-2078). We utilized the KRAB domain found between amino acids 1 and 97 of the zinc finger protein KOX1 (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). In this case an N-terminal fusion with a zinc-finger polypeptide is constructed. Finally, to explore the utility of histone deacetylation for repression, amino acids 1 to 36 of the Mad mSIN3 interaction domain (SID) are fused to the N-terminus of the zinc finger protein (Ayer, D. E., Laherty, C. D., Lawrence, Q. A., Armstrong, A. P. & Eisenman, R. N. (1996) Mol. Cell. Biol. 16, 5772-5781). This small domain is found at the N-terminus of the transcription factor Mad and is responsible for mediating its transcriptional repression by interacting with mSIN3, which in turn interacts the co-repressor N-CoR and with the histone deacetylase mRPD1 (Heinzel, T., Lavinsky, R. M., Mullen, T.-M., S\check{s}derstr\check{s}m, M., Laherty, C. D., Torchia, J., Yang, W.-M., Brard, G., Ngo, S. D. & al., e. (1997) Nature 387, 43-46). To examine gene-specific activation, transcriptional activators are generated by fusing the zinc finger polypeptide to amino acids 413 to 489 of the herpes simplex virus VP16 protein (Sadowski, I., Ma, J., Triezenberg, S. & Ptashne, M. (1988) Nature 335, 563-564), or to an artificial tetrameric repeat of VP16's minimal activation domain, (Seipel, K., Georgiev, O. & Schaffner, W. (1992) EMBO J. 11, 4961-4968), termed VP64. The transcription regulating domains can be operatively linked to a DNA binding domain at either the N— or C-terminus of the binding domain.

A transcription regulating domain, when present, can be situated at either the N— or C-terminal of a present polypeptide or adjacent to and between a DNA binding domain and a linker (see FIG. 2). A polypeptide of this invention can contain one or more transcription regulating domains. Where a plurality of transcription regulating domains are present, each domain can be the same or different. Similarly, a single polypeptide can contain both repressor and activation domains. FIG. 2 shows an exemplary polypeptides of this invention having two DNA binding domains and either a single repressor or single activation domain or a combination of such repressor and activation domains.

III. Polynucleotides and Expression Vectors

The invention includes a nucleotide sequence encoding a zinc finger-nucleotide binding polypeptide. DNA sequences encoding the zinc finger-nucleotide binding polypeptides of the invention, including native, truncated, and expanded polypeptides, can be obtained by several methods. For example, the DNA can be isolated using hybridization procedures which are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR). RNA sequences of the invention can be obtained by methods known in the art (See, for example, Current Protocols in Molecular Biology, Ausubel, et al., Eds., 1989).

The development of specific DNA sequences encoding zinc finger-nucleotide binding polypeptides of the invention can be obtained by: (1) isolation of a double-stranded DNA sequence from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA. Of these three methods for developing specific DNA sequences for use in recombinant procedures, the isolation of genomic DNA is the least common. This is especially true when it is desirable to obtain the microbial expression of mammalian polypeptides due to the presence of introns.

For obtaining zinc finger derived-DNA binding polypeptides, the synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucleic Acid Research 11:2325, 1983).

IV. Pharmaceutical Compositions

In another aspect, the present invention provides a pharmaceutical composition comprising a therapeutically effective amount of a polypeptide of this invention or a therapeutically effective amount of a nucleotide sequence that encodes such a polypeptide in combination with a pharmaceutically acceptable carrier.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the composition.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, as well as pH buffering agents and the like which enhance the effectiveness of the active ingredient.

The therapeutic pharmaceutical composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

V. Uses

In one embodiment, a method of the invention includes a process for modulating (inhibiting or suppressing) expression of a nucleotide sequence comprising a binding motif, which method includes the step of contacting the binding motif with an effective amount of a subject polypeptide that binds to the motif. The binding motif is preferably located in a transcriptional control region of the target gene. A transcriptional control region is any region of a gene involved in regulating transcription. An exemplary such region is a promoter. In the case where the nucleotide sequence is a promoter, the method includes inhibiting the transcriptional transactivation of a gene containing a zinc finger-DNA binding motif. The term “inhibiting” refers to the suppression of the level of activation of transcription of a structural gene containing a zinc finger-nucleotide binding motif, for example. In addition, the gene transcription regulating polypeptide may bind a motif within a structural gene or within an RNA sequence.

The term “effective amount” includes that amount which results in the deactivation of a previously activated promoter or that amount which results in the inactivation of a promoter containing a zinc finger-nucleotide binding motif, or that amount which blocks transcription of a structural gene or translation of RNA. The amount of gene transcription regulating polypeptide required is that amount necessary to either displace a native zinc finger-nucleotide binding protein in an existing protein/promoter complex, or that amount necessary to compete with the native zinc finger-nucleotide binding protein to form a complex with the promoter itself. Similarly, the amount required to block a structural gene or RNA is that amount which binds to and blocks RNA polymerase from reading through on the gene or that amount which inhibits translation, respectively. Preferably, the method is performed intracellularly. By functionally inactivating a promoter or structural gene, transcription or translation is suppressed. Delivery of an effective amount of the inhibitory protein for binding to or “contacting” the cellular nucleotide sequence containing the zinc finger-nucleotide binding protein motif, can be accomplished by one of the mechanisms described herein, such as by retroviral vectors or liposomes, or other methods well known in the art.

The term “modulating” refers to the suppression, enhancement or induction of a function. For example, the gene transcription regulating polypeptide of the invention may modulate a promoter sequence by binding to a motif within the promoter, thereby enhancing or suppressing transcription of a gene operatively linked to the promoter nucleotide sequence. Alternatively, modulation may include inhibition of transcription of a gene where the gene transcription regulating polypeptide binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript.

The promoter region of a gene includes the regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA to RNA. In most cases the resulting RNA molecule serves as a template for synthesis of a specific protein; sometimes RNA itself is the final product.

The promoter region may be a normal cellular promoter or, for example, an onco-promoter. An onco-promoter is generally a virus-derived promoter. For example, the long terminal repeat (LTR) of retroviruses is a promoter region which may be a target for a zinc finger binding polypeptide variant of the invention. Promoters from members of the Lentivirus group, which include such pathogens as human T-cell lymphotrophic virus (HTLV) 1 and 2, or human immunodeficiency virus (HIV) 1 or 2, are examples of viral promoter regions which may be targeted for transcriptional modulation by a polypeptide of the invention.

The Examples that follow show the use of polypeptides of this invention to alter expression of polynucleotides encoding particular gene products. The Examples are representative of particular embodiments of this invention and are not limiting of the specification and/or claims in any way.

### EXAMPLE 1

### Transcription Factor Polypeptides

A series of transcription factor polypeptides that target specific genes were made and used to alter expression of gene products. E2c and E2x are six finger proteins that bind in the post-transcriptional and pre-translatorial region of the erbB2 gene, as fusion proteins with the effector domains vp64 and SKD they regulate erbB2 expression in both directions [Beerli, R. et al: PNAS (1998), 95, 14628-14633; and Dreier, B. et al: J Biol Chem Aug. 3, 2001; 276(31):29466-78]. E3 and E3Y are six finger proteins that bind in the post transcriptional and pre-translatorial region of the erbB3 gene, as fusion proteins with the effector domains vp64 and SKD they regulate erbB3 expression in both directions [Beerli, R. et al: PNAS(1998), 95, 14628-14633; and Dreier, B. et al: J Biol Chem Aug. 3, 2001; 276(31):29466-78]. Exemplary polypeptides are shown below together with the DNA target sequence for that polypeptide. The six finger proteins were generated as described elsewhere (Segal, D et al: PNAS (1999), 96,2758-2763).

E2cJ15E3Y

(SEQ ID NO:6)

MAQAALEPGEKPYACPECGKSFSRKDSLVRHQRTHTGEKPYKCPECGKSF
SQSGDLRRHQRTHTGEKPYKCPECGKSFSDCRDLARHQRTHTGEKPYACP
ECGKSFSQSSHLVRHQRTHTGEKPYKCPECGKSFSDCRDLARHQRTHTGE
KPYKCPECGKSFSRSDKLVRHQRTHTGGGGSGGGGTGEKPYACPECGKSF
SDKKDLTRHQRTHTGEKPYKCPECGKSFSDCRDLARHQRTHTGEKPYKCP
ECGKSFSQLLAHLRAHQRTHTGEKPYACPECGKSFSQSGDLRRHQRTHTG
EKPYKCPECGKSFSRSDNLVRHQRTHTGEKPYKCPECGKSFSDPGALRVH
QRTHTGKKTSGQAG
DNA TARGET SEQUENCE:

(SEQ ID NO:7)

E2c: GGG GCC GGA GCC GCA GTG;

(SEQ ID NO:8)

E3Y: ATC GAG GCA AGA GCC ACC
E2xJ15E3

(SEQ ID NO:9)

MAQAALEPGEKPYACPECGKSFSQSSHLVRHQRTHTGEKPYKCPECGKSF
SRSDHLAEHQRTHTGEKPYKCPECGKSFSDKKDLTRHQRTHTGEKPYACP
ECGKSFSQSSNLVRHQRTHTGEKPYKCPECGKSFSQSSHLVRHQRTHTGE
KPYKCPECGKSFSDKKDLTRHQRTHTHTGGGGSGGGGTGEKPYACPECGK
SFSDPGALVRHQRTHTGEKPYKCPECGKSFSQSSHLVRHQRTHTGEKPYK
CPECGKSFSDCRDLARHQRTHTGEKPYACPECGKSFSQSSHLVRHQRTHT
GEKPYKCPECGKSFSDCRDLARHQRTHTGEKPYKCPECGKSFSQSSHLVR
HQRTHTGKKTSGQAG
DNA TARGET SEQUENCE:

(SEQ ID NO:10)

E3: GGA GCC GGA GCC GGA GTC;

(SEQ ID NO:11)

E2X: ACC GGA GAA ACC AGG GGA
E3J15E2x

(SEQ ID NO:12)

MAQAALEPGEKPYACPECGKSFSDPGALVRHQRTHTGEKPYKCPECGKSF
SQSSHLVRHQRTHTGEKPYKCPECGKSFSDCRDLARHQRTHTGEKPYACP
ECGKSFSQSSHLVRHQRTHTGEKPYKCPECGKSFSDCRDLARHQRTHTGE
KPYKCPECGKSFSQSSHLVRHQRTHTGGGGSGGGGTGEKPYACPECGKSF
SQSSHLVRHQRTHTGEKPYKCPECGKSFSRSDHLAEHQRTHTGEKPYKCP
ECGKSFSDKKDLTRHQRTHTGEKPYACPECGKSFSQSSNLVRHQRTHTGE
KPYKCPECGKSFSQSSHLVRHQRTHTGEKPYKCPECGKSFSDKKDLTRHQ
RTHTGKKTSGQAG
DNA TARGET SEQUENCE:

(SEQ ID NO:10)

E3: GGA GCC GGA GCC GGA GTC;

(SEQ ID NO:13)

E2X: ACC GGA GAA ACC AGG GGA
E2cJ15E3

(SEQ D NO:14)

MAQAALEPGEKPYACPECGKSFSRKDSLVRHQRTHTGEKPYKCPECGKSF
SQSGDLRRHQRTHTGEKPYKCPECGKSFSDCRDLARHQRTHTGEKPYACP
ECGKSFSQSSHLVRHQRTHTGEKPYKCPECGKSFSDCRDLARHQRTHTGE
KPYKCPECGKSFSRSDKLVRHQRTHTGGGGSGGGGTGEKPYACPECGKSF
SDPGALVRHQRTHTGEKPYKCPECGKSFSQSSHLVRHQRTHTGEKPYKCP
ECGKSFSDCRDLARHQRTHTGEKPYACPECGKSFSQSSHLVRHQRTHTGE
KPYKCPECGKSFSDCRDLARHQRTHTGEKPYKCPECGKSFSQSSHLVRHQ
RTHTGKKTSGQAG
DNA TARGET SEQUENCE:

(SEQ ID NO:7)

E2c: GGG GCC GGA GCC GCA GTG;

(SEQ ID NO:10)

E3: GGA GCC GGA GCC GGA GTC
E2xJ15E3y

(SEQ ID NO:15)

MAQAALEPGEKPYACPECGKSFSQSSHLVRHQRTHTGEKPYKCPECGKSF
SRSDHLAEHQRTHTGEKPYKCPECGKSFSDKKDLTRHQRTHTGEKPYACP
ECGKSFSQSSNLVRHQRTHTGEKPYKCPECGKSFSQSSHLVRHQRTHTGE
KPYKCPECGKSFSDKKDLTRHQRTHTHTGGGGSGGGGTGEKPYACPECGK
SFSDKKDLTRHQRTHTGEKPYKCPECGKSFSDCRDLARHQRTHTGEKPYK
CPECGKSFSQLAHLRAHQRTHTGEKPYACPECGKSFSQSGDLRRHQRTHT
GEKPYKCPECGKSFSRSDNLVRHQRTHTGEKPYKCPECGKSFSDPGALRV
HQRTHTGKKTSGQAG
DNA TARGET SEQUENCE:

(SEQ ID NO:13)

E2X: ACC GGA GAA ACC AGG GGA;

(SEQ ID NO:8)

E3Y: ATC GAG GCA AGA GCC ACC

Various combinations of E2c, E2x, E3 and E3y (linked with short or long linkers) were attached to either a repressor domain (SKD) or activation domain (VP64). The charts below summarize such polypeptide.

Short Linker Constructs:J15

| Effector domain | Zif1 | Zif2 |
|---|---|---|
| SKD | E2c | E3 |
| SKD | E2c | E3y |
| SKD | E2x | E3y |
| SKD | E3 | E2x |

| Zif1 | Zif2 | Effector domain |
|---|---|---|
| E2c | E3 | Vp64 |
| E2c | E3y | Vp64 |
| E2x | E3y | Vp64 |
| E3 | E2x | Vp64 |

Long Linker Constructs: J30

| Effector domain | Zif1 | Zif2 |
|---|---|---|
| SKD | E2c | E3 |
| SKD | E2c | E2x |
| SKD | E2c | E3y |
| SKD | E3 | E3y |
| SKD | E3 | E2x |
| SKD | E2x | E3y |
| SKD | E2x | E3 |

| Zif1 | Zif2 | Effector domain |
|---|---|---|
| E2c | E3 | Vp64 |
| E2c | E3y | Vp64 |
| E2x | E3y | Vp64 |
| E2x | E3 | Vp64 |
| E3 | E2x | Vp64 |

Instead of the regular canonical linker between Zif domains (TGEKP) the twelve fingers contain longer linkers to connect the six finger proteins. They were introduced by PCR using the forward primers J15F or J30F and pMalseq Back as reverse primer. The PCR template can be a regular three or six finger in pMal (See Scheme 1, below):

Sequence Primer Short Linker J15:

```
Sequence Range: 1 to 90
                      >SmaI
                |
      >XmaI|
          | |
          | | 10 20 30
TGA GCC CGG GGG CGG TGG CTC GGG CGG TGG
ACT CGG GCC CCC GCC ACC GAG CCC GCC ACC
      >BsrFI >BglII
          | |
      >AgeI >XbaI |
          | | |
          | 40 | | 50 60
TGG GAC CGG TTC CTC TAG ATC TTC CTC CAC
ACC CTG GCC AAG GAG ATC TAG AAG GAG CTG
                      70 80
CGG GGA GAA GCC CTA TGC TTG TCC GGA
CCC CCT CTT CGG GAT ACG AAC AGG CCT
  90
ATG (SEQ ID NO:16)
TAC (SEQ ID NO:17)
```

Sequence Primer Long Linker J30F:

```
Sequence Range: 1 to 99
  >XmaI
|
| 10 20 30
CCC GGG TCC TCT GGT GGC GGT GGC TCG GGC
GGG CCC AGG AGA CCA CCG CCA CCG AGC CCG
                      40 50 60
GGT GGT GGG GGT GGT TCC ACT GGC GGT GGC
CCA CCA CCC CCA CCA AGG TGA CCG CCA CCG
                                    >AgeI
                                        |
                      70 | 80 90
TCG GGC GGT GGT GGG ACC GGT TCC TCT AGA
AGC CCG CCA CCA CCC TGG CCA AGG AGA TCT
TCT TCC TCC (SEQ ID NO:18)
AGA AGG AGG (SEQ ID NO:19)
```

[image]

The PCR Product was cleaved with XmaI and SpeI and any original Zif protein cleaved with AgeI and SpeI. The long linker containing zinc finger was then inserted between AgeI and SpeI. XmaI/AgeI form compatible cohesive ends and both restriction sites (XmaI/AgeI) disappear during that cloning step. A new finger can be inserted by cutting this construct AgeI/SpeI and the original finger XmaI/SpeI. Thus the resulting twelve finger has the same restriction sites as a six finger (Scheme 2). Consequently the assembly can be extended to n fingers, and can also combine three with six fingers etc.

[image]

### EXAMPLE 2

### Binding to Target DNA and Transcription Regulation

Polypeptides from Example 1 were tested for their binding to specific DNA target sequences and for their ability to alter transcription. The results are summarized below.

The 12 finger fusion proteins between e2c/e2x and e3/E3y are able to regulate both genes at once. This hypothesis was tested by transfecting the different pMXSKD-12 finger and pMX12finger vp64 constructs in 293-Gag-Pol cells and infecting A431 cells with the resulting virus. Three days after infection the cells were harvested and analyzed for erbB2 and erbB3 expression levels by FACS. This procedure was done as described previously (Segal, D et al: PNAS(1999), 96,2758-2763). ELISA data of raw extracts of e2cJ15/30E3 and e2cJ15/30e3y show that all four constructs bind their respective targets.

Seven different constructs have been analyzed for regulatory effects on erbB2 and erbB3. Down regulation of both erbB2 and erbB3 to basal levels was observed for pMXSKDE2cJ15E3. Most efficient up regulation of both erbB2 and erbB3 was observed with the construct pMX E2cJ15E3vp64, pMX E2xJ15E3vp64 also affects both genes, as well as e2cE3yvp64.

pMXe2cJ15e3 repressed erbB2 and erbB3 down to basal levels. The other three constructs work worse, they just affect erbB3 expression without repressing erbB2. This is true for both, e2x, which has lower affinity to its target (Km=15 nM), and the high affinity (Km=0.5 nM) e2c containing constructs. As e2x is once on the N-terminus, and once on the C-terminus, different positioning within the twelve finger does not improve erbB2 repression. This is not a question of zinc finger protein expression levels, as estimated by GFP expression. pMXe2cJ15e3 is one of the weaker expressors and the most effective repressor.

Also for the activators, pMXe2cJ15e3vp64 showed the best effect by clearly activating erbB2 and erbB3. In contrast to the repressors, however, the two other constructs also activated both genes. ErbB3 seems to be activated a bit stronger compared to erbB2.

Double targeting within one promoter could increase the overall weak activation effect of zinc fingers. For that purpose pcDNAe2cJ15e2xvp64 and pcDNASKDe2cJ15e2x were transiently transfected in Hela cells, together with the Luciferase reporter construct E2p. 36 fold repression was observed for SKDe2c compared to 8 fold repression for SKDe2cJ15e2x. For activation, 45 fold activation was observed for the twelve finger compared to 78 fold activation by vp64e2c.

The twelve finger construct pMXe2cJ15CD144#5 does activate erbB2 but not CD144 in A431 cells. Two independent clones were tested and showed the same effect. One clone was fully sequenced and just one aa of the last helix was unreadable or ambiguous.

Overview of repressor domains tested with erbB2

| | Repression of erbB2 |
|---|---|
| | (as measured in |
| | transient reporter assay, |
| Repression Domain | Beerli et al.(1998) |
| SKD (entire KRAB-domain) | >90% |
| SID (mSin3 interaction domain) | 80% |
| ERD | 50% |
| hCIR | cloned, not tested |
| none (ZIF alone) | 30% |

### EXAMPLE 3

### General Procedures

Construction of Zinc Finger Library and Selection via Phage Display

Construction of the zinc finger library was based on the C-7 protein (U.S. Pat. No. 6,140,081). Finger 3 recognizing the 5′-GCG-3′ subsite was replaced by a domain binding to a 5′-GAT-3′ subsite via a PCR overlap strategy using a primer coding for finger 3 (5′-GAG-GAAGTTTGCCACCAGTGGCAACCTGGTGAGGCATACCAAAATC-3′)(SEQ ID NO:20) and a vector-specific primer (5′-GTAAAACGACGGCCAGTGCCAAGC-3′)(SEQ ID NO:21). Randomization of the zinc finger library by PCR overlap extension was essentially as is well known in the art. The library was ligated into the phagemid vector pComb3H. Growth and precipitation of phage were performed using standard techniques. Binding reactions were performed in a volume of 500 ml of zinc buffer A (ZBA: 10 mM Tris, pH 7.5, 90 mM KCl, 1 mM MgCl₂, 90 mM ZnCl2 ), 0.2% bovine serum albumin, 5 mM dithiothreitol, 1% Blotto (Bio-Rad), 20 mg of double-stranded, sheared herring sperm DNA containing 100 ml of precipitated phage (10¹³ colony-forming units). Phage were allowed to bind to non-biotinylated competitor oligonucleotides for 1 h at 4° C. before the biotinylated target oligonucleotide was added. Binding continued overnight at 4° C. After incubation with 50 ml of streptavidin-coated magnetic beads (Dynal; blocked with 5% Blotto in ZBA) for 1 h, beads were washed 10 times with 500 ml of ZBA, 2% Tween 20, 5 mM dithiothreitol, and once with buffer containing no Tween. Elution of bound phage was performed by incubation in 25 ml of trypsin (10 mg/ml) in Tris-buffered saline for 30 min at room temperature.

Hairpin competitor oligonucleotides had the sequence 5′-GGCCGCN′N′N′AT CGAGTTTTCTCGATNNNGCGGCC-3′ (SEQ ID NO:22), where NNN represents the finger-2 subsite oligonucleotides and N′N′N′ its complementary bases. Target oligonucleotides were biotinylated and usually added at 72 nM in the first three rounds of selection and then decreased to 36 and 18 nM in the sixth and last round. As competitor a 5′-TGG-3′ finger-2 subsite oligonucleotide was used to compete with the parental clone. An equimolar mixture of 15 finger-2 5′-ANN-3′ subsites, except for the target site and competitor mixtures of each finger-2 subsites of the type 5′-CNN-3′, 5′-GNN-3′, and 5′-TNN-3′ were added in increasing amounts with each successive round of selection. Usually no specific 5′-ANN-3′ competitor mix was added in the first round.

Multitarget Specificity Assay and Gel Mobility Shift Analysis—The zinc finger-coding sequence was subcloned from pComb3H into a modified bacterial expression vector pMal-c2 (New England Biolabs). After transformation into XL1-Blue (Stratagene) the zinc finger-maltose-binding protein (MBP) fusions were expressed by addition of 1 nM isopropyl b-D-thiogalactoside (IPTG). Freeze/thaw extracts of these bacterial cultures were applied in 1:2 serial dilutions to 96-well plates coated with streptavidin (Pierce) and were tested for DNA binding specificity against each of the 16 5′-GAT ANN GCG-3′ target sites. Enzyme-linked immunosorbent assay (ELISA) was performed. After incubation with a mouse anti-MBP antibody (Sigma, 1:1000), a goat anti-mouse antibody coupled with alkaline phosphatase (Sigma, 1:1000) was applied. Detection occurred by addition of alkaline phosphatase substrate (Sigma), and the A405 was determined by a microtiter plate reader with SOFTMAX2.35 (Molecular Devices). Gel shift analysis was performed with purified protein (Protein Fusion and Purification System, New England Biolabs).

Site-Directed Mutagenesis of Finger 2—Finger-2 mutants were constructed by PCR. As PCR template the pMal vector encoding for C7.GAT was used. PCR products containing a mutagenized finger 2 and 5′-GAT-3′ finger 3 were subcloned via NsiI and SpeI restriction sites in frame with finger 1 of C7 (5′-GCG-3′) into a modified pMal-c2 vector (New England Biolabs).

Construction of Polydactyl Zinc Finger Proteins—Three-finger proteins were constructed by finger-2 stitchery using the SP1C framework. The proteins generated in this work contained helices recognizing 5′-GNN-3′ DNA sequences, as well as 5′-ANN-3′ and 5′-TAG-3′ helices. Six finger proteins were assembled via compatible XmaI and BsrFI restriction sites. Analysis of DNA-binding properties were performed using freeze/thaw extracts from IPTG-induced bacteria. For the analysis of the capability of these proteins to regulate gene expression, they were fused to the activation domain VP64 or repression domain KRAB of Kox-1; VP64 (tetrameric repeat of the herpes simplex virus VP16 minimal activation domain) and subcloned into pcDNA3 (Invitrogen) or the retroviral pMX-IRES-GFP vector) internal ribosome-entry site (IRES) and green fluorescent protein (GFP).

Transfection and Luciferase Assays—HeLa cells were used at a confluency of 40-60%. Cells were transfected with 160 ng of reporter plasmid (pGL3; Promega) containing the promoter sequence with zinc finger-binding sites and 40 ng of effector plasmid (zinc finger-effector domain fusions in pcDNA3) in 24-well plates. Cell extracts were prepared 48 h after transfection and measured with luciferase assay reagent (Promega) in a MicroLumat LB96P luminometer (EG & Berthold, Gaithersburg, Md.).

Retroviral Gene Targeting and Flow Cytometric—As primary antibody an ErbB-1-specific mAb EGFR (Santa Cruz Biotechnology), ErbB-2-specific mAb FSP77 (gift from Nancy E. Hynes), and an ErbB-3-specific mAb SGP1 (Oncogene Research Products) were used. Fluorescently labeled donkey F(ab9)2 anti-mouse IgG was used as secondary antibody (Jackson ImmunoResearch).

Bacterial Extracts of pMal-Fusion Proteins for ELISA Assays

The selected zinc finger proteins were cloned into the pMal vector (New England Biolabs) for expression. The constructs were transferred into the E. coli strain XL1-Blue by electroporation and streaked on LB plates containing 503 g/ml carbenecillin. Four single colonies of each mutant were inoculated into 3 ml of SB media containing 50 3 g/ml carbenecillin and 1% glycose. Cultures were grown overnight at 37° C. 1.2 ml of the cultures were transformed into 20 ml of fresh SB media containing 50 3 g/ml carbenecillin, 0.2% glycose, 90 3 g/ml ZnCl₂ and grown at 37° C. for another 2 hours. IPTG was added to a final concentration of 0.3 mM. Incubation was continued for 2 hours. The cultures were centrifuged at 4° C. for 5 minutes at 3500 rpm in a Beckman GPR centrifuge. Bacterial pellets were resuspended in 1.2 ml of Zinc Buffer A containing 5 mM fresh DTT. Protein extracts were isolated by freeze/thaw procedure using dry ice/ethanol and warm water. This procedure was repeated 6 times. Samples were centrifuged at 4° C. for 5 minutes in an Eppendorf centrifuge. The supernatant was transferred to a clean 1.5 ml centrifuge tube and used for the ELISA assays.

ELISA Assays—Finger-2 variants of C7.GAT were subcloned into bacterial expression vector as fusion with maltose-binding protein (MBP) and proteins were expressed by induction with 1 mM IPTG (proteins (p) are given the name of the finger-2 subsite against which they were selected). Proteins were tested by enzyme-linked immunosorbent assay (ELISA) against each of the 16 finger-2 subsites of the type 5′-GAT CNN GCG-3′ to investigate their DNA-binding specificity.

In addition, the 5′-nucleotide recognition was analyzed by exposing zinc finger proteins to the specific target oligonucleotide and three subsites which differed only in the 5′-nucleotide of the middle triplet. For example, pCAA was tested on 5′-AAA-3′, 5′-CAA-3′, 5′-GAA-3′, and 5′-TAA-3′ subsites. Many of the tested 3-finger proteins showed exquisite DNA-binding specificity for the finger-2 subsite against they were selected.

What is claimed is:

1. A non-naturally occurring transcription factor polypeptide comprising a plurality of DNA binding domains operatively linked with a flexible peptide linker having from 5 to 50 amino acid residue sequences.

2. The polypeptide of claim 1 that contains 2 or 3 DNA binding domains.

3. The polypeptide of claim 1 wherein the linker has from 5 to 30 amino acid residues.

4. The polypeptide of claim 1 wherein the linker has from 5 to 15 amino acid residue sequences.

5. The polypeptide of claim 1 further comprising a transcription regulating factor.

6. The polypeptide of claim 5 wherein the transcription regulating factor represses transcription.

7. The polypeptide of claim 5 wherein the transcription regulating factor activates transcription.

8. The polypeptide of claim 5 wherein the transcription regulating factor is located at the N-terminus of the polypeptide.

9. The polypeptide of claim 5 wherein the transcription regulating factor is located at the C-terminus of the polypeptide.

10. The polypeptide of claim 1 further comprising a plurality of transcription regulating factors.

11. The polypeptide of claim 1 wherein each DNA binding domain contains from 3 to 6 zinc finger DNA binding peptides.

12. The polypeptide of claim 11 wherein each DNA binding domain contains 6 zinc finger DNA binding peptides.

13. The polypeptide of claim 12 further comprising one or more transcription regulating domains.

14. A polynucleotide that encodes the polypeptide of claim 1.

15. An expression vector that contains the polynucleotide of claim 14.

16. A cell transfected with the polynucleotide of claim 14.

17. A process of altering expression of a nucleotide sequence containing a binding motif, comprising the step of contacting the binding motif with an effective amount of a polypeptide of claim 1 that binds to the motif.

18. A process of simultaneously altering expression of a first nucleotide sequence containing a first binding motif and a second nucleotide sequence that contains a second binding motif, the process comprising the step of contacting the binding motifs with an effective amount of a polypeptide of claim 1 that binds to both the first and second binding motif.
